(19) United States
(12) Patent Application Publication
(10) Pub. No.: US 2005/0272685 A1
(43) Pub. Date: Dec. 8, 2005

(54) Intraductal treatment targeting methylated promoters in breast cancer

(76) Inventor: David Hung, Belmont, CA (US)

Correspondence Address: CYTYC CORPORATION, 250 CAMPUS DRIVE, MARLBOROUGH, MA, 01752, US

(21) Appl. No.: 11/203,800

(22) Filed: Aug. 15, 2005

Related U.S. Application Data
(63) Continuation of application No. 10/101,631, filed on Mar. 21, 2002, now abandoned.
(60) Provisional application No. 60/279,762, filed on Mar. 30, 2001.

(51) Int. Cl. A61K 48/00

(52) U.S. Cl. 514/44

(57) ABSTRACT

The invention provides compositions for targeting methylated promoters in breast cancer and pre-cancer patients. The invention also provides methods for intraductal treatment of breast cancer and pre-cancer patients by delivering intraductally an agent that targets methylated promosters of silenced genes.

This application claims the benefit of U.S. Provisional Application 60/279,762, David Hung, filed Mar. 30, 2001.

### FIELD OF THE INVENTION

The field of this invention is methods and compositions to intraductally treat breast cancer and precancer by targeting methylated promoters of breast cancer related genes.

### BACKGROUND

Some genes that have lower expression in breast cancer than normal tissue are silenced by hypermethylation of promoter sequences of the gene.

Hypermethylation, the creation of 5-methylacytosines, occurs in the CpG rich regions (called CpG islands) of the promoter. The CpG island sequences consist minimally of a CCGG sequence. Hypermethylation of these regions appears to suppress transcription and is associated with cancer. The methylation specific PCR (MSP) detects the 5-methyl cytosine moieties the presence of which indicates hypermethylation.

Transcription of the downstream gene is inactivated by promoter hypermethylation. Thus, in a cancer context, a gene controlled by a hypermethylated promoter becomes silent in the cancer or malignant state, and silenced to a lesser degree in an atypical or precancer state.

Expression of these genes or silencing of them is evidenced by the methylation specific PCR of ductal epithelial cells from which these genes can be expressed in a normal healthy individual. Transcription of a hypermethylated gene can be restored upon demethylation of the CpG islands in the gene's promoter.

### SUMMARY OF THE INVENTION

An aspect of the invention is a composition to reduce or eliminate hypermethylation of promoters controlling breast cancer-related genes. Accordingly is provided, a composition for intraductal administration to a patient having abnormal breast ductal epithelial cells including atypical or malignant cells comprising a demethylating agent selected from the group consisting of an inhibitor of DNA methylation, a demethylating agent, an antagonist of DNA methyl transferase activity, and a deliverable amount of a biocompatible solution suitable as a vehicle for the agent for delivery intraductally to the patient in order to contact breast duct epithelial cells therein.

An aspect of the invention is a method of treating a patient having locally identified hypermethylation of promoters controlling breast cancer-related genes. Accordingly, is provided a method of treating a patient having a breast duct comprising atypical or malignant breast duct epithelial cells comprising, delivering intraductally to the breast duct an amount of an agent comprising an agent selected from the group consisting of an inhibitor of DNA methylation, a demethylating agent, and an antagonist of DNA methyl transferase activity sufficient to inhibit or reverse DNA methylation of genes transcribed within said breast duct epithelial cells.

### DETAILED DESCRIPTION OF PREFERRED EMBODIMENTS OF THE INVENTION

The following preferred embodiments and examples are offered by way of illustration and not by way of limitation.

The invention is a novel composition and treatment method for treating patients having breast cancer and precancer conditions that have been identified in a specific breast duct or ducts of the patient.

The composition comprises one or more of a demethylating agent (to remove existing hypermethylations), an inhibitor of DNA methylation (e.g. an agent comprising a moiety that competitively binds methyl groups and/or prevents methylation at cytosines) or an antagonist/inhibitor of DNA methyl transferase (the enzyme) or its activity leading to methylation of cytosines. DNA methyl transferase (MeTase or DMT) is the enzyme that catalyzes the methylation reaction at cytosines. One DMT is e.g. 5-aza-2-deoxycytidine (5-aza-CdR). Antisense oligonucleotides against the region of the promoter comprising a CpG island may also be used as an inhibitor of DNA methylation. The composition may comprise one or more or all or several of these classes of agents that relate to and/or affect methylation or demethylation at CpG sites on promoters for breast cancer-related genes. Antagonists or inhibitors can be any molecule capable of antagonizing or inhibiting the target bio-activity. Thus, for example, antagonists or inhibitors can be for example small organic molecules, proteins, polypeptides, peptides, oligonucleotides, lipids, carbohydrates, polymers and the like.

The composition also necessarily requires an agent or medium that makes the active agent deliverable intraductally to a breast duct. The composition comprising the agent or medium for delivery to a breast duct and comprising one or more of the classes of active agents listed need be biocompatible for humans, optimally provides an optimal delivery window of the active agent to resident ductal epithelial cells in the breast duct. Thus, also the composition can comprise a solution that comprises saline or other common safe deliverable solutions or agents or mediums; e.g. the composition having an agent or medium to aid the intraductal delivery can optimally comprise a viscous or gel material or other such medium that may provide the active agent carried by the medium a longer residence and/or activity in the duct to which is it delivered.

Breast cancer genes that are known to be vulnerable to hypermethylation and subsequent degrees of gene transcription and expression silencing include, e.g. cyclin D2, RARbeta2, twist, BRCA1, maspin, estrogen receptor, progesterone receptor, and e-cadherin. There are others not listed here. Other genes having promoters that can be methylated but that are not necessarily present in a breast context include e.g. p16 (INK4a), P 15 (INK4b), P 14 (ARF), death associated protein (DAP), retinoblastoma Rb, and von-Hippel-Lindaur (VHL) gene.

The treatment method comprises delivering the claimed composition intraductally to a breast duct in a patient. Preferably the duct has been previously identified as having premalignant (e.g. hyperplastic and/or atypical) or malignant (carcinoma) cells and thus been identified as a target for the local treatment protocol proposed in the method.

The treatment method comprises delivering intraductally to a breast duct (e.g. a target duct previously identified as having atypical or malignant cells) an amount of an agent such as an inhibition of DNA methylation, a demethylating agent, and/or an antagonist of DNA methyl-transferase. The delivery to the duct can be accomplished by accessing a breast duct with a delivery tool (e.g. a catheter, cannula, or the like) and infusing the agent (in a suitable medium or solution for delivery of the active agent) into the duct to contact target ductal epithelial cells lining the duct. The delivery can also be accomplished e.g. by pump delivery, time-release capsule placed in the duct, and the like.

The amount of agent can vary, but in any event optimally will be an amount sufficient to target all atypical or malignant cells in the duct. Estimates of the quantity of target cells can be made upon the initial identification of the target duct (e.g. by cytological evaluation of ductal epithelial cells retrieved from the duct. The amount may vary depending on the agent's potency and other mitigating factors such as the extent of any time delay of delivery of the agent once inside the duct (e.g. with a time release formulation). Other factors such as whether the ductal epithelial cells are atypical or malignant (e.g. greater therapeutic activity may be needed for malignant cells), and/or how many genes might be affected by the methylation activity can also affect a determination of the amount of active agent to deliver to any given duct. The agent should be delivered in a sufficient amount to inhibit or reverse DNA methylation on promoters controlling genes transcribed and/or expressed in ductal epithelial cells of the target breast duct. Preferably the status of ductal markers and of the ductal epithelial cells will be evaluated prior to intraductal delivery of the demethylating and/or antimethylating agent(s), e.g. the evaluation can comprise MSP of the methylated genes (e.g to identify them and/or to quantify the amount of methylation) and/or cytological evaluation of the ductal epithelial cells (e.g. identify hyperplastic, atypical, or malignant cells).

### EXAMPLE

A breast duct on the right breast of a patient is identified as having atypical cells that are suspicious for malignancy. Four genes are tested in ductal epithelial cells retrieved from a breast duct by methylation specific PCR (MSP) to further establish a methylated state of some promoters of some genes transcribed and/or expressed in the ductal environment. The information quantified as a degree of methylation in addition helps to determine an amount or specific activity required of the agent for effective treatment. It is found that RARbeta2, twist, maspin, and cyclin D2 are all genes expressed in the ductal epithelium that show some percentage of methylation on the promoter CpG islands as indicated by MSP. A viscous composition of mixture of a demethylating agent, an antisense oligonucleotide against CpG regions on the various promoters of the various target genes, and an inhibitor of DNA methyl transferase (5-azaCdR) activity are administered. The formula provides a week-long time-release of the active agents present in the composition. Ductal fluid is again retrieved and analyzed a month following the procedure in order to assess a need for follow-up 2nd dose intraductal administration of agent by conducting cytological analysis of retrieved ductal epithelial cells, and by MSP of the genes studied and targeted in the first administration.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-11. (canceled)

12. A method for inhibiting DNA methylation of breast cancer genes in a patient having premalignant or malignant breast duct epithelial cells in a breast duct, the method comprising: delivering a composition into the breast duct of the patient, said composition comprising an inhibitor of DNA methylation and a biocompatible solution suitable as a vehicle for delivering the inhibitor of DNA methylation into the breast duct of the patient; thereby inhibiting DNA methylation of breast cancer-related genes within said premalignant or malignant breast duct epithelial cells.

13. (canceled)

14. The method of claim 12 wherein the breast cancer genes are selected from the group consisting of cyclin D2, RARbeta2, twist, BRCA1, maspin, estrogen receptor, progesterone receptor, e-cadherin, p16 (INK4a), P15 (INK4b), P14 (ARF), death associated protein (DAP), retinoblastoma Rb, and vonHippel-Lindaur (VHL) gene.

15. (canceled)

16. (canceled)

17. The method of claim 1 12 wherein the DNA methylation inhibitor competitively binds methyl groups and prevents methylation at cytosines.

18. The method of claim 1 12 wherein the DNA methylation inhibitor is an oligonucleotide directed against a CpG island region of a promoter of a breast cancer related gene.

19. (canceled)

20. (canceled)

21. The method of claim 12 wherein the biocompatible solution is selected from the group consisting of saline, viscous material, and gel material.
